(19) United States
(12) Patent Application Publication
(10) Pub. No.: US 2004/0225344 A1
(43) Pub. Date: Nov. 11, 2004

(54) Devices, kits and methods for placing multiple intraluminal medical devices in a body vessel

(76) Inventors: Andrew K. Hoffa, Bloomington, IN (US); Jacob A. Flagle, Bloomington, IN (US); Michael L. Garrison, Indianapolis, IN (US)

Correspondence Address: FRASER MARTIN BUCHANAN MILLER LLC, 132C WEST SECOND STREET, PERRYSBURG, OH, 43551-1401, US

(21) Appl. No.: 10/831,564

(22) Filed: Apr. 23, 2004

Related U.S. Application Data
(60) Provisional application No. 60/465,197, filed on Apr. 23, 2003.

(51) Int. Cl. A61F 2/06

(52) U.S. Cl. 623/1.1

(57) ABSTRACT

Devices, kits, and methods for delivering multiple intraluminal medical devices into a body vessel are provided.

### CROSS-REFERENCE TO RELATED APPLICATIONS

This application claims priority to U.S. Provisional Application Ser. No. 60/465,197, filed on Apr. 23, 2003, the entire disclosure of which is hereby incorporated into this disclosure in its entirety.

### FIELD OF THE INVENTION

The present invention relates to methods of delivering multiple intraluminal medical devices into a body vessel. In preferred embodiments, the invention relates to methods of delivering multiple prosthetic venous valves into a body vessel. The invention also relates to medical devices and kits for use in the methods of the invention. The invention further relates to methods of supplying intraluminal medical devices.

### BACKGROUND OF THE INVENTION

Minimally invasive techniques and instruments for placement of intraluminal medical devices have developed over recent years. A wide variety of treatment devices that utilize minimally invasive technology has been developed and includes stents, stent grafts, occlusion devices, infusion catheters and the like. Minimally invasive intravascular devices have especially become popular with the introduction of coronary stents to the U.S. market in the early 1990's. Coronary and peripheral stents have been proven to provide a superior means of maintaining vessel patency, and have become widely accepted in the medical community. Furthermore, the use of stents has been extended to treat aneurysms and to provide occlusion devices, among other uses.

Typically, intraluminal medical devices, such as stents, are deployed at a point of treatment in a body vessel by a delivery device that has been directed through the body vessel. Once the intraluminal device has been deployed at the point of treatment, the delivery device is withdrawn from the vessel. Using prior art devices and methods, it is necessary to insert a second delivery device following removal of the first if deployment of an additional intraluminal device is desired. Each additional intraluminal medical device for deployment in the vessel necessitates an additional delivery device. Each additional delivery device, like the first, must be directed through the body vessel to a point of treatment, which increases the length and complexity of the procedure.

Recently, prosthetic venous valves have developed in the art. These valves are designed to replace the function of incompetent natural valves. Considering the presence of multiple natural valves along the length of a body vessel, it may be necessary in some treatments to deliver multiple prosthetic venous valves into a single vessel. The prior art does not, however, contain any devices or methods that allow for the delivery of multiple intraluminal medical devices without the need for retracting a first delivery device from the vessel and inserting a second delivery device into the vessel for each additional intraluminal device being delivered.

Therefore, there is a need for medical devices and various methods that allow for the delivery of multiple intraluminal medical devices into a body vessel without the need for retracting a delivery device, such as a sheath, from the vessel and inserting a second delivery device into the vessel between deployments.

### SUMMARY OF THE INVENTION

The present invention provides devices, kits, and methods for placing multiple intraluminal medical devices into a body vessel. The methods include providing a delivery medical device, such as a sheath that defines a lumen, and a plurality of intraluminal medical devices, such as prosthetic venous valves. Next, the methods include advancing the delivery medical device through the body vessel and advancing a first intraluminal medical device through the lumen of the delivery device. Next, the method includes deploying a first intraluminal medical device at a first point of treatment. After deployment of the first medical device, a second medical device is advanced to a second point of treatment and deployed. Between the deployment of the first medical device and the advancement of the second medical device, the delivery device is not removed entirely from the body vessel.

In a preferred embodiment, a method according to the invention comprises advancing a sheath defining a lumen into a body vessel, advancing a first prosthetic venous valve through the lumen, deploying the first prosthetic venous valve, advancing a second prosthetic venous valve through the lumen without removing the sheath from the body vessel, and deploying the second prosthetic venous valve.

In a particularly preferred embodiment, a method according to the present invention comprises inserting a sheath into a body vessel at an insertion point, advancing the sheath to a first point of treatment in the body vessel, deploying a first prosthetic venous valve, retracting the sheath to a second point of treatment, advancing a second prosthetic venous valve through the sheath, and deploying the second prosthetic venous valve. The first and second prosthetic venous valves are deployed from the sheath without removing the sheath completely from the body vessel.

The present invention also provides medical devices for use in the methods of the present invention. In particular, the present invention provides medical devices that facilitate the advancement of additional intraluminal medical devices into a body vessel following the deployment of an initial intraluminal medical device without the removal of an installed delivery medical device. The medical devices of the present invention comprise carriers that include an intraluminal medical device. In one embodiment, the medical device comprises an elongate member with an intraluminal medical device disposed on a distal end thereof. The elongate member is adapted for insertion into the lumen of a delivery device, such as the sheath. Preferably, the elongate member further includes a jacket member disposed around the intraluminal medical device. Particularly preferably, the jacket comprises a peel-away sheath.

In another embodiment, the medical device comprises a carrier that defines an internal passage. In each carrier of this type, an intraluminal medical device is disposed in the interior passage. The carrier can define a connector that is adapted to form a mating connection with another connector on the delivery device.

The present invention also comprises kits useful in the methods of the invention. The kits include a delivery device, such as a sheath defining a lumen, and a plurality of medical devices in accordance with the present invention. In preferred embodiments, the plurality of medical devices comprises a plurality of elongate members each having a prosthetic venous valve disposed on a distal tip thereof. Each of the plurality of elongate members further includes a jacket member disposed around the intraluminal medical device, such as a peel-away sheath. In a particularly preferred embodiment, the kit includes a sheath having a first elongate member disposed therein, and at least a second elongate member free of the sheath. At least one elongate member includes a jacket member disposed around the associated intraluminal medical device. In further preferred embodiments, the kit can include additional elongate members that are free of the sheath. These additional elongate members also preferably include jacket members disposed around the associated intraluminal medical devices.

In another embodiment, a kit according to the present invention comprises a sheath defining a lumen and a plurality of housing members. The sheath preferably defines a first connector. Each of the housing members defines an interior passage and preferably defines a second connector that is adapted for mating with the first connector. An intraluminal medical device, such as a prosthetic venous valve, is disposed in the interior passage of each of the plurality of housing members. Particularly preferably, kits according to the embodiment of the present invention further include an obturator adapted for advancing an intraluminal medical device through the interior passage of a housing member and into the lumen of the sheath.

The present invention also provides methods of supplying intraluminal medical devices for use in treating human and veterinary patients in which it is desirable to deploy multiple intraluminal medical devices in a body vessel. In a preferred embodiment, the method of supplying according to the present invention comprises supplying a sheath defining a lumen with a plurality of carriers. Each of the plurality of carriers includes an intraluminal medical device and is capable of being operably associated with the sheath. Preferably, the sheath, plurality of carriers and intraluminal medical devices are supplied as a kit.

### BRIEF DESCRIPTION OF THE DRAWINGS

FIG. 1 is a side view, partially broken away, of a medical device for use in the methods of the invention.

FIG. 2 is a side view, partially broken away, of a medical device according to one embodiment of the invention.

FIG. 3 is a side view of a kit and its components according to one embodiment of the invention.

FIG. 4 is a side view, partially broken away, of a medical device according to another embodiment of the invention.

FIG. 5 is a side view, partially broken away, of an obturator for use with the medical device illustrated in FIG. 4.

FIG. 6 is a wide view of a kit and its components according to another embodiment of the invention.

FIG. 7 is a block diagram illustrating a method according to one embodiment of the invention.

FIG. 8 is a block diagram illustrating a method according to another embodiment of the invention.

FIG. 9 is a block diagram illustrating a method according to another embodiment of the invention.

### DETAILED DESCRIPTION OF EXEMPLARY EMBODIMENTS OF THE INVENTION

Additional understanding of the invention can be obtained by reference to the following detailed description of exemplary embodiments in conjunction with review of the appended drawings. The detailed description and drawings are intended merely to describe exemplary embodiments of the invention, and are in no way intended to limit the scope of the invention. Rather, the detailed description and drawings serve to enable one of ordinary skill in the relevant art to make and use the invention.

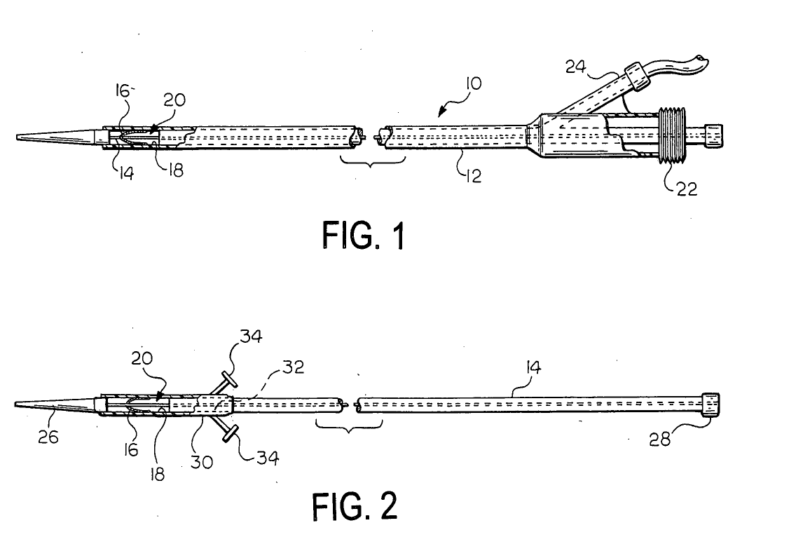

FIG. 1 illustrates a medical device 10 for use in the methods of the invention. The medical device 10 comprises a sheath 12, a dilator 14, and an intraluminal medical device 16. The sheath 12 defines a lumen 18 and the dilator 14 is disposed therein. The intraluminal medical device 16 is disposed on the dilator 14 at a chamber section 20. The sheath 12 can further include a connector 22, which will be described more fully below, and various other functional components, such as access port 24.

FIG. 2 illustrates a carrier medical device in accordance with one embodiment of the invention. In this embodiment, the carrier comprises the dilator 14 illustrated in FIG. 1 and jacket member 30 disposed around the chamber area 20 and intraluminal medical device 16. In this embodiment, the carrier 14 is an elongate member having distal 26 and proximal 28 ends. The chamber area 20 is disposed near the distal end 26 and the intraluminal medical device 16 is disposed in the chamber section 20. As illustrated in FIG. 2, the jacket member 30 preferably comprises a sheath that surrounds the carrier 14 at the chamber end 20. In the illustrated embodiment, the jacket member 30 comprises a peel-away sheath. Peel-away sheath 30 includes one or more weakened areas 32 and tabs 34. The peel-away sheath 30 is removed by pulling tabs 34 at the weakened areas 32. After tearing down the length of the sheath 30, the peel-away sheath 30 is free of the carrier 14, and the chamber area 20 and intraluminal medical device 16 are exposed.

The jacket member 30 preferably has a length that, at a minimum, extends along a length of an intraluminal medical device 16 in the chamber area. At a maximum, the length of the jacket member 30 can be the length of the carrier 14. Preferably, however, the jacket member 30 has a length that, when the member 30 is axially moved to a position at the proximal end of the carrier 14 by way of the carrier 14 being inserted into the sheath 12, prevents the distal tip 26 of the carrier 14 from extending on a distal end of the sheath 12 prior to removal of the jacket member 30. This avoids unintentional exposure of the chamber section 20 or deployment of the intraluminal medical device 16 before such exposure and/or deployment is desired.

The carrier 14 is placed into a sheath 12 and advanced along the lumen 18 of the sheath until intraluminal medical device 16 is positioned at a desired point. The peel-away sheath 30 is preferably slidably mounted on the carrier 14. This facilitates insertion of the carrier 14 into the sheath 12 while minimizing the potential for unintended release of the intraluminal medical device 16 from the chamber section 20 prior to insertion into the lumen 18. As is known in the art, the intraluminal medical device can comprise a self-expanding stent, and premature removal of a constraining force can lead to unintended deployment of the device.

In use, the distal end 26 of the carrier 14 is inserted into the lumen 18 of the sheath 12. As the distal end 26 is advanced further into the lumen 18, the peel-away sheath 30 is forced towards the proximal end 28 of the carrier 14 by the sheath 12. This ensures that intraluminal medical device 16 is restrained throughout the process of inserting carrier 14 into sheath 12, initially by the jacket member 30, and eventually by the sheath 12.

The carrier 14 is suitable for use in the methods of the present invention as it provides a medical device that can be inserted into the lumen 18 of sheath 12 and subsequently removed. Additional carriers 14 can then be advanced into the lumen 18 to deploy additional intraluminal medical devices. Any suitable number of additional carriers 14 can be sequentially advanced into the lumen 18 of the sheath 12. The previously inserted carrier need only be removed from the lumen 18 prior to insertion of the next carrier 14.

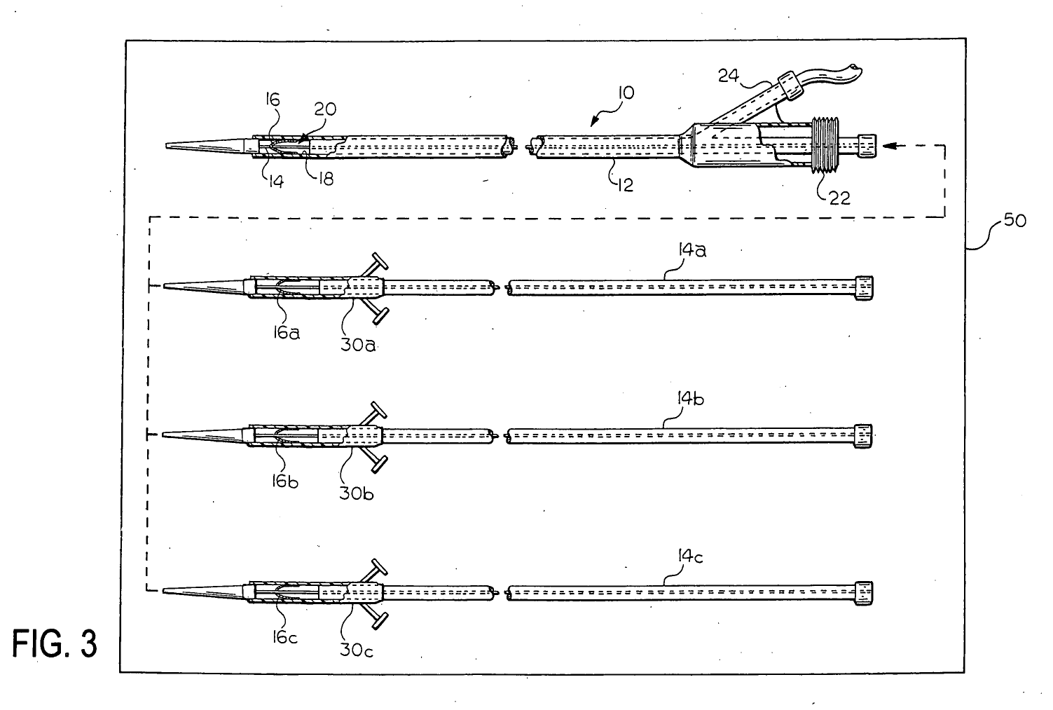

Together, the sheath 12 and multiple carriers 14 provide a suitable kit for delivering a plurality of intraluminal medical devices into the body vessel. FIG. 3 illustrates the components of a kit 50 according to one embodiment of the invention. In this embodiment, the kit 50 includes a delivery medical device, such as sheath 12, and multiple carriers 14a, 14b, 14c. Each of the carriers 14a, 14b, 14c includes an intraluminal medical device 16a, 16b, 16c and preferably includes a jacket, such as peel-away sheath 30a, 30b, 30c. Further, each of the carriers 14a, 14b, 14c are capable of being operably associated with the sheath 12, such as being adapted to be inserted into the lumen 18 of sheath 12. Preferably, as illustrated in FIG. 3, the kit 50 includes a sheath 12 having a first carrier 14 disposed in the lumen 18. In this configuration, the kit 50 allows for an initial use of the device assembly 10 to deploy the first intraluminal medical device 16, and subsequent use of additional carriers 14a, 14b, 14c to sequentially deploy additional intraluminal devices 16a, 16b, 16c. Additional carriers 14a, 14b, 14c are preferably supplied free of the sheath 12, i.e., not within the lumen 18 of the sheath 12. Also, as described above, each of the additional carriers 14a, 14b, 14c, preferably includes a jacket member 30a, 30b, 30c.

FIG. 4 illustrates a carrier 60 according to another embodiment of the invention. Carrier 60 comprises a housing member 62 that defines an interior passage 64. Intraluminal medical device 16 is disposed in the interior passage 64. Housing member 62 preferably defines a connector 66 that is adapted to form a mating connection to a connector on a delivery device, such as connector 22 on sheath 12 illustrated in FIG. 1.

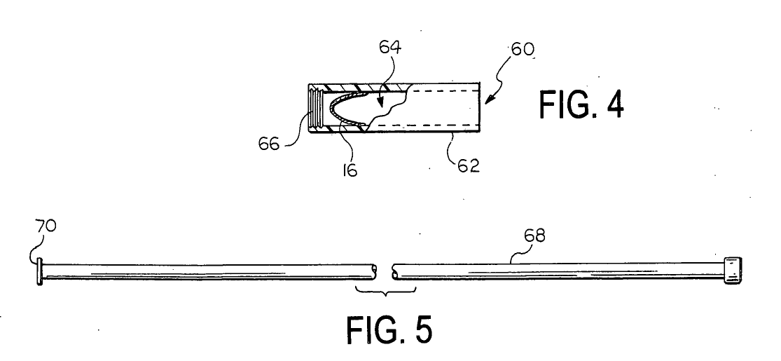

The intraluminal medical device 16 associated with carrier 16 is deployed by advancing the device 16 out of the interior passage 64. An obturator 68, illustrated in FIG. 5, provides a pushing surface 70 for accomplishing this movement. The pushing surface 70 is preferably adapted to slidably move within the interior passage 64 of the housing 62 while advancing the intraluminal medical device 16 through the interior passage 64.

The carrier 60 is particularly well-suited for facilitating the introduction of multiple intraluminal medical devices 16 into a delivery device, such as sheath 12, without necessitating the removal of the sheath 12 from a body vessel. For example, the connector 66 can be mated with a connector 22 on the sheath 12 to define a continuous lumen between the sheath 12 and housing member 62. The continuous lumen comprises the lumen 18 of the sheath and the interior passage 64 of the carrier 60. Once the carrier is connected to the sheath 12, obturator 68, via pushing surface 70, can be used to advance intraluminal medical device 16 out of the interior passage 64 of the carrier 60 and into the lumen 18 of the sheath 12. Due to the connection between the connectors 66, 22, the intraluminal medical device is restricted throughout this process and no unintended deployment occurs during the transition from the carrier 60 to the sheath 12.

The connectors 22, 66 can be any suitable mating pair of connectors known to those skilled in the art. Examples of suitable connectors include mating threaded connectors, mating clamping connectors, mating luer lock fittings, and the like. In one embodiment, the connector on the sheath 12 comprises a valve, such as a silicone iris or check valve, that receives housing member 62. The housing member 62 in this embodiment, therefore, does not define any structural connector. Rather, the form of the housing member 62 itself forms the connector 66. Indeed, in this embodiment, housing member 62 preferably has a smooth outer surface. Thus, in this embodiment, the housing member 62 is inserted into the valve on the sheath 12 to form a connection between the sheath 12 and carrier 60, and to define the desired continuous lumen.

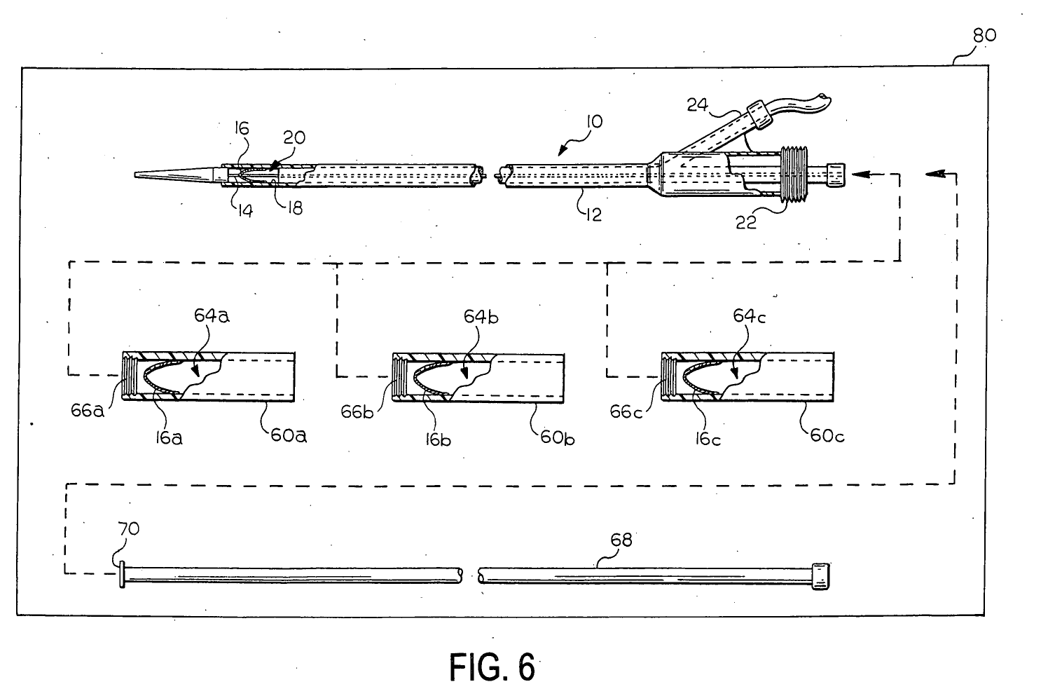

FIG. 6 illustrates the components of a kit 80 according to another embodiment of the invention. The kit 80 includes a delivery device, such as sheath 12, that defines a lumen 18 and a plurality of carriers 60a, 60b, 60c. Each of the carriers 60a, 60b, 60c includes an intraluminal medical device 16a, 16b, 16c. Further, each carrier 60a, 60b, 60c preferably includes a connector 66a, 66b, 66c individually adapted to form a mating connection to a connector 22 on sheath 12. The kit 80 further includes an obturator 68 having a pushing surface 70 adapted to advance intraluminal medical devices 16a, 16b, 16c out of interior passage 64a, 64b, 64c of the carrier 60a, 60b, 60c. The several intraluminal medical devices 16a, 16b, 16c can be sequentially inserted into the lumen 18 of the sheath 12 by first forming a connection between the connector 66a of the first carrier 60a and the connector 22 of the sheath 12 and advancing the intraluminal medical device 16a out of the interior passage 64a into the lumen 18. Next, the obturator 68 can be advanced through the lumen 18 of the sheath 12 until the intraluminal medical device 16s is deployed. Next, the obturator 68 is removed from the lumen 18 of the sheath 12 and the first carrier 60a is removed from the sheath 12. The second carrier 60b is connected to the connector 22 in similar fashion as the first carrier 60a. Following connection, the second intraluminal medical device 16b is advanced out of the interior passage 64b and into the lumen 18 by the obturator 68. Again, after deployment of the second intraluminal medical device 16b, the third intraluminal medical device 16c can be deployed in similar fashion.

While the kits 50, 80 illustrated herein each contain three additional carriers, it is contemplated that any suitable number of additional carriers can be used. The actual number chosen will depend on several factors, including the number of intraluminal medical devices to be deployed in any single body vessel.

As illustrated in FIG. 6, the kit preferably includes an elongate carrier 14 initially disposed in a sheath 12. The elongate carrier 14 includes a first intraluminal medical device 16 in a chamber region 20. This configuration of the kit 80 facilitates efficient deployment of a first intraluminal medical device 16 and deployment of subsequent intraluminal medical devices 16a, 16b, 16c following removal of elongate carrier 14.

In all embodiments of the present invention, the intraluminal medical device can comprise any suitable intraluminal medical device, such as a stent, an occluder, a filter, and a prosthetic venous valve. The intraluminal medical device can comprise a self-expanding or balloon expandable device. Examples of suitable stents for use in the present invention include those described in U.S. Pat. No. 6,464,720 to Boatman et al. for a RADIALLY EXPANDABLE STENT; U.S. Pat. No. 6,231,598 to Berry et al. for a RADIALLY EXPANDABLE STENT; U.S. Pat. No. 6,299,635 to Frantzen for a RADIALLY EXPANDABLE NON-AXIALLY CONTRACTING SURGICAL STENT; and U.S. Pat. No. 4,580,568 to Gianturco for a PERCUTANEOUS ENDOVASCULAR STENT AND METHOD FOR INSERTION THEREOF. In exemplary embodiments of the invention, the intraluminal medical device comprises a prosthetic valve, such as a prosthetic venous valve. Any suitable prosthetic valve can be utilized in the devices and methods according to the present invention. Examples of suitable prosthetic venous valves include those described in U.S. Pat. No. 6,508,833 to Pavcnik et al. for a MULTIPLE-SIDED INTRALUMINAL MEDICAL DEVICE, and published United States Patent Application 2001/0039450 to Pavcnik et al. for an IMPLANTABLE MEDICAL DEVICE. Other suitable prosthetic venous valves include stentless prosthetic venous valves, such as the valves described in commonly-owned Provisional Patent Application Ser. No. 60/459,475, filed on Apr. 1, 2003, and entitled Percutaneously Deployed Vascular Valve With Wall-Adherent Adaptations. Each of these references are hereby incorporated into this disclosure in their entirety for the express purpose of describing suitable implantable medical devices for use in and with the devices, kits, and methods according to the present invention.

It may be desirable to deploy different types of intraluminal medical devices in a single procedure and/or vessel. For example, it may be desirable to deploy a prosthetic venous valve at one location in a vessel, and deploy a self-expandable stent at another location in the same vessel. Thus, any suitable combination of intraluminal medical devices can be used in the kits and methods of the present invention. The exact combination and number of intraluminal medical devices used in any particular method or included in any particular kit will depend on various factors, including the condition being treated.

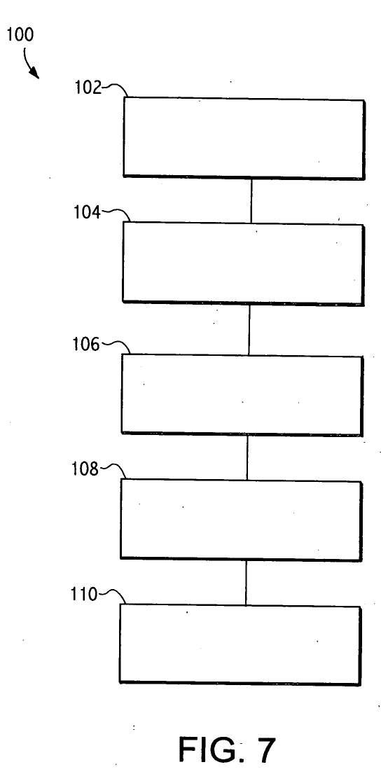

FIG. 7 illustrates a method 100 of delivering multiple medical devices into a body vessel according to one embodiment of the invention. In a first step 102, a first intraluminal medical device is advanced to a first point of treatment (POT). Preferably, this step comprises advancing a delivery assembly, such as assembly 10 illustrated in FIG. 1, that includes a sheath and a carrier including the intraluminal medical device through a body vessel. Alternatively, this step can comprise advancing an intraluminal medical device through a sheath that has previously been inserted into the body vessel. Further, the intraluminal medical device can be disposed on a carrier, or can be advanced through the sheath via an obturator.

In another step 104, the first intraluminal medical device is deployed. The manner in which this step is accomplished will depend on the arrangement of the intraluminal medical device within the delivery device. For example, if the intraluminal medical device is disposed on a elongate carrier, such as carrier 14 illustrated in FIGS. 1-3, the intraluminal medical device can be deployed by withdrawing the sheath to expose the intraluminal medical device. If, however, the intraluminal medical device is not disposed on an elongate carrier member and is simply contained within a lumen of the delivery device independent of a carrier, the intraluminal medical device can be deployed simply by forcing the medical device out of an end of a lumen. For example, if the intraluminal medical device is contained within a carrier, such as carrier 60 illustrated in FIGS. 4 and 6, an obturator, such as obturator 68 illustrated in FIG. 5, can be used to force the intraluminal medical device out of a delivery device.

In another step 106, a second intraluminal medical device is advanced through the delivery device. This step is accomplished after deployment of the first intraluminal medical device, and the manner in which this step is accomplished will also depend upon the configuration of the second intraluminal medical device. For example, if an elongate carrier is utilized, the elongate carrier is advanced through the lumen of the delivery device. However, if a housing carrier is utilized, the intraluminal medical device is advanced through the lumen of the delivery device via an obturator. Preferably, the advancement of the second intraluminal medical device is accomplished only after removal of any advancement means used to advance the first intraluminal medical device, such an elongate carrier or an obturator.

In another step 108, a second intraluminal medical device is positioned at a second POT in the body vessel. In another step 110, the second intraluminal medical device is deployed. Again, the mechanism of deploying the second intraluminal medical device will depend on the configuration of the medical device and carrier, as described above.

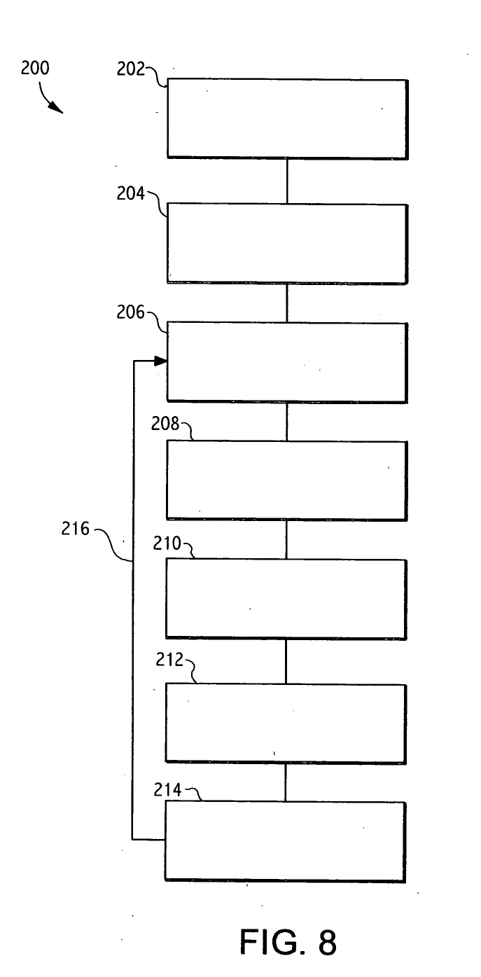

FIG. 8 illustrates a method 200 according to another embodiment of the invention. In the method according to this embodiment of the invention, a first step 202 comprises advancing a first prosthetic venous valve to a first POT of a body vessel. In another step 204, the first prosthetic venous valve is deployed. In another step 206, the carrier, such as the dilator 14 illustrated in FIG. 1, is removed from the body vessel. In another step 208, a second carrier with a second prosthetic venous valve is inserted into the delivery device. In another step 210, the second prosthetic venous valve is advanced through the delivery device. In another step 212, the second prosthetic venous valve is positioned at a second point of treatment. In another step 214, the second prosthetic venous valve is deployed. In a repeating step 216, the steps between the removing the carrier step 206 through the deploying the second prosthetic venous valve step 214, inclusively, can be repeated as many times as necessary. The number of repetitions chosen will depend on the number of prosthetic venous valves desired to be delivered.

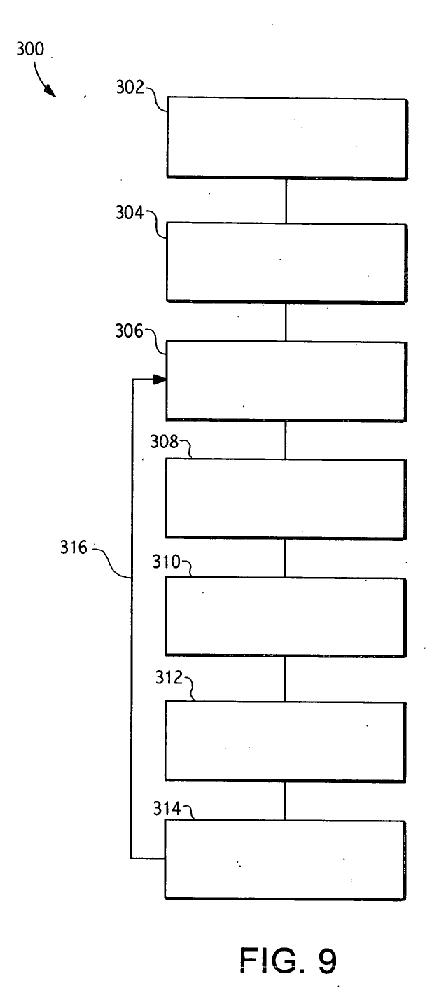

FIG. 9 illustrates a method 300 according to another embodiment of the present invention. In this embodiment, the method 300 comprises a first step 302 of advancing a first prosthetic venous valve to a first POT. In another step 304, the first prosthetic venous valve is deployed. In another step 306, the advancer member associated with the first prosthetic venous valve is removed. As used herein, the term “advancer member” refers to a carrier, such as an elongated carrier 14 illustrated in FIG. 2, or an obturator, such as the obturator 68 illustrated in FIG. 5 or other suitable member used to advance a prosthetic venous valve through a delivery device.

In another step 308, a second prosthetic venous valve is inserted into the delivery device. In this embodiment, the insertion step 308 is preferably accomplished by connecting a carrier containing the second prosthetic venous valve, such as carrier 60 illustrated in FIG. 4, to the delivery device and advancing the second prosthetic venous valve through the carrier and into the delivery device. In another step 310, the second prosthetic venous valve is advanced through the delivery device. In another step 312, the second prosthetic venous valve is positioned at a second POT. In another step 314, the second prosthetic venous valve is deployed. In a repeating step 316, the steps between and including the removal of the advancer 306 and the deploying of the second prosthetic venous valve 314 are repeated any suitable number of times. The actual number of repetitions 316 chosen will depend on the desired number of prosthetic venous valves to be deployed.

The present invention also provides a method of supplying intraluminal medical devices for use in methods of treating human or veterinary patients in which it is desirable to deploy multiple intraluminal medical devices in a body vessel. In one embodiment, this method of the invention comprises supplying a sheath defining a lumen with a plurality of carriers. Each of the carriers includes an intraluminal medical device and is capable of being operably associated with the sheath. As used herein, the term “operably associated” refers to a formation of a connection between the carrier and the sheath to define a continuous path of travel for an intraluminal medical device from the carrier into the sheath. Preferably, the sheath and plurality of carriers and intraluminal medical devices are supplied as kits.

In exemplary embodiments, the intraluminal medical devices comprise prosthetic venous valves. Also preferable, the carriers comprise medical devices in accordance with the present invention, such as carrier 14 illustrated in FIG. 2 and carrier 60 illustrated in FIG. 4. If the carriers supplied comprise housings defining interior passages, such as the carrier 60 illustrated in FIG. 4, the method of supplying according to the present invention also preferably includes supplying an obturator adapted for advancing an intraluminal medical device through the housing member and into the lumen of the sheath.

The foregoing disclosure includes the best mode of the inventor for practicing the invention. It is apparent, however, that those skilled in the relevant art will recognize variations of the invention that are not described herein. While the invention is defined by the appended claims, the invention is not limited to the literal meaning of the claims, but also includes these variations.

1. A method of delivering multiple medical devices into a body vessel, comprising:
providing a sheath that defines a lumen;
providing a plurality of medical devices capable of being advanced through the lumen;
advancing the sheath through said body vessel;
advancing a first medical device of the plurality of medical devices through the lumen;
deploying the first medical device at a first point of treatment in said body vessel;
advancing a second medical device of the plurality of medical devices through the lumen;
deploying the second medical device at a second point of treatment in said body vessel.

2. The method of claim 1, wherein the first medical device is disposed within the sheath to form a delivery assembly, and wherein advancing the sheath and advancing a first medical device comprise advancing the delivery assembly through said body vessel.

3. The method of claim 1, wherein advancing a second medical device comprises advancing a dilator having the second medical device disposed thereon.

4. The method of claim 1, wherein advancing a second medical device comprises connecting a housing member containing the second medical device to the sheath and advancing the second medical device out of the housing member and into the sheath.

5. The method of claim 1, wherein each of the plurality of medical devices comprises a prosthetic valve.

6. The method of claim 5, wherein each of the plurality of medical devices comprises a prosthetic venous valve.

7. The method of claim 1, wherein the first medical device comprises a prosthetic valve and the second medical device comprises a stent.

8. The method of claim 1, wherein the first medical device comprises a stent and the second medical device comprises a prosthetic valve.

9. A method of implanting multiple prosthetic venous valves in a body vessel, comprising advancing a sheath defining a lumen into said body vessel, advancing a first prosthetic valve through the lumen, and advancing a second prosthetic valve through the lumen without removing the sheath from said vessel.

10. A method of implanting a plurality of medical devices at a plurality of points of treatment in a body vessel, comprising:
inserting a sheath defining a lumen into said body vessel at an insertion point;
advancing the sheath to a first point of treatment of said plurality of points of treatment;
deploying a first medical device of said plurality of medical devices;
retracting the sheath to a second point of treatment of said plurality of points of treatment;
advancing a second medical device of said plurality of medical devices through the lumen; and
deploying the second medical device of said plurality of medical devices.

11. The method of claim 10, wherein the first and second medical devices each comprise a prosthetic valve.

12. The method of claim 11, wherein the first and second medical devices each comprise a prosthetic venous valve.

13. The method of claim 10, wherein the first medical device comprises a prosthetic valve and the second medical device comprises a stent.

14. The method of claim 10, wherein the first medical device comprises a stent and the second medical device comprises a prosthetic valve.

15. The method of claim 10, wherein advancing a second medical device comprises advancing a dilator having the second medical device disposed thereon.

16. The method of claim 10, wherein advancing a second medical device comprises connecting a housing member containing the second medical device to the sheath and advancing the second medical device out of the housing member and into the sheath.

17. A kit for implanting a plurality of intraluminal medical devices, comprising:
a sheath defining a lumen; and
a plurality of carriers, each of the plurality of carriers including one of said plurality of intraluminal medical devices and capable of being operably associated with the sheath.

18. A kit according to claim 17, wherein the plurality of carriers comprises a plurality of elongate members, each of the plurality of carriers having one of said plurality of intraluminal medical devices disposed thereon.

19. A kit according to claim 18, wherein each of the plurality of elongate members includes a jacket member disposed around the one of said plurality of intraluminal medical devices.

20. A kit according to claim 17, wherein the plurality of carriers comprises a plurality of housing members, each of the plurality of housing members defining a passage and having one of said plurality of intraluminal medical devices disposed in the passage.

21. A kit according to claim 20, wherein the sheath defines a first connector and each of the plurality of housing members defines a second connector adapted for mating with the first connector.

22. A kit according to claim 20, further comprising an obturator adapted for advancing each of said plurality of intraluminal medical devices through one of said plurality of housing members and into the lumen of the sheath.

23. A kit according to claim 17, wherein each of said plurality of medical devices comprises a prosthetic valve.

24. A kit according to claim 23, wherein each of said plurality of medical devices comprises a prosthetic venous valve.

25. A kit according to claim 17, wherein one of said plurality of medical devices comprises a stent and one of said plurality of medical devices comprises a prosthetic valve.
